(19) United States
(12) Patent Application Publication
(10) Pub. No.: US 2026/0241137 A1
(43) Pub. Date: Aug. 20, 2026

(54) DEVICE FOR DISINFECTING AND CLEANING A CATHETER, AND A METHOD THEREOF

(71) Applicant: GAMBRO LUNDIA AB, Lund (SE)

(72) Inventor: Raj M R APORVA, Shivamogga (IN)

(21) Appl. No.: 18/872,148

(22) PCT Filed: May 30, 2023
(86) PCT No.: PCT/EP2023/064371

§ 371 Date: Dec. 5, 2024

(30) Foreign Application Priority Data
Jun. 8, 2022 (IN) 202241032764

(51) Int. Cl. A61M 25/00; A61L 2/18; A61L 103/15; B08B 9/032

(52) U.S. Cl. CPC A61M 25/00; A61L 2/18; B08B 9/0321; A61L 2103/15; A61L 2202/17; A61M 2025/0019; B08B 2209/032

(57) ABSTRACT

A device for disinfecting a catheter, and a method thereof is described. The device comprises a stem, and an expandable member fixed at a front portion of the stem The expandable member is wetted with a disinfectant. A sheath assembly houses the stem and the expandable member, and includes a front end and a plurality of slots present near the front end The slots allow for outward expansion of the expandable member when the expandable member is pushed towards the front end by operating the stem. When the sheath assembly is inserted within the catheter and the expandable member is brought outward by pushing through the stem, the expandable member comes in contact with inner surface of the catheter, thereby disinfecting the catheter.

### FIELD OF INVENTION

The present invention generally relates to field of catheters used in medical applications, and more specifically, relates to a device for disinfecting and cleaning a catheter.

### BACKGROUND OF THE INVENTION

The subject matter discussed in the background section should not be assumed to be prior art merely as a result of its mention in the background section. Similarly, a problem mentioned in the background section are associated with the subject matter of the background section should not be assumed to have been previously recognized in the prior art. The subject matter in the background section merely represents different approaches, which in and of themselves may also correspond to implementations of the claimed technology.

Catheters are thin and flexible tubes with varying levels of stiffness depending on their application. Catheters are inserted in a body cavity, duct, vessel, skin, or adipose tissues, to treat diseases or to perform a surgical procedure. By modifying the structure and material of the catheters, it is possible to tailor catheters for cardiovascular, urological, gastrointestinal, neurovascular, and ophthalmic applications. Catheters allow drainage, administration of fluids or gases, or access of a body cavity.

Central Venous Catheters (CVCs), Peripherally Inserted Central Catheters (PICCs) and Peritoneal dialysis (PD) Catheters are indispensible devices used vastly in medical practice, particularly among critically ill patients, cancer patients, and those receiving intravenous feeding. These catheters allow for the rapid infusion of concentrated medications, fluids, or blood products that otherwise cannot be administered into peripherally extreme veins via standard intravenous probes.

Although these catheters (CVCs, PICCs and PD catheters) provide necessary vascular access, they also put the patients at risk of significant infection, which can be life-threatening. Most Catheter-Related Bloodstream Infections (CRBI) are associated with endoluminal contamination and subsequent colonization of the catheters during their long-term usage. For instance, a PD catheter is prone to develop biofilm, fibrin sheath, bacterial build-up, and scarring formation at inner diameter because of continuous presence of fluid. A catheter affected with such factors causes thrombosis and peritonitis in patients. Generally, a catheter becomes contaminated when micro-organisms settle over its external surface by coming in contact with skin of a patient or an operator, tracheostomy secretions, wounds, and the like. A contaminated catheter can lead to Central Line-Associated Bloodstream Infection (CLABSI), a serious infection that occurs when germs, usually bacteria or viruses, enter the bloodstream of a patient through a contaminated catheter. Such infections also require unplanned removal of contaminated catheters.

Efforts have been made in the past to disinfect catheters using stringent infection control practices. Some of these stringent infection control practices include usage of fluids to disinfect a catheter each time after its usage. A typical example is Citra-lock™ which is a solution prefilled into the catheter after every procedure, as an antibacterial, anticoagulant, and anti-biofilm fluid. However, such techniques are not effective for sterilization and disinfection of harmful micro-organisms settled inside and biofilms deposited inside the catheter.

Currently available catheters are designed primarily to ensure a tight connection with an intravenous tubing, but are not designed specifically to prevent contamination, such as endoluminal catheter microbial colonization.

Therefore, there exists a need for techniques and devices that are effective in removal of microbial contamination of a catheter. The present invention satisfies this need and envisages to provide related advantages as well.

### OBJECTS OF THE INVENTION

An objective of the present invention is to provide a device for disinfecting and cleaning a catheter, and a method thereof.

Another objective of the present invention is to provide a device capable of preventing bloodstream infections in patients, and a method thereof.

Another objective of the present invention is to provide a device and a method capable of disinfecting inner surface of a catheter and removing microbial contamination.

Still another objective of the present invention is to provide a device to preventing formation of fibrin sheath inside the catheter, and a method thereof.

### SUMMARY OF THE INVENTION

An aspect of the present invention relates to a device for disinfecting and cleaning inner surface of a catheter to remove microbial contamination and prevent formation of fibrin sheath in the catheter. The device includes a stem, and an expandable member fixed at a front portion of the stem, the expandable member wetted with a disinfectant. The device also includes a sheath assembly housing the stem and the expandable member. The sheath assembly includes a front end that is closed, and a plurality of slots present near the front end.

The plurality of slots allow for outward expansion of the expandable member when the expandable member is pushed towards the front end by the stem, and allow for inward movement of the expandable member when the expandable member is pushed away from the front end by the stem. When the sheath assembly is inserted within the catheter and the expandable member is brought outward by pushing through the stem, the expandable member wetted with the disinfectant comes in contact with inner surface of the catheter, thereby disinfecting the catheter.

In one aspect, the expandable member is selected from a group consisting of a sponge, a foam, and cotton.

In one aspect, the front end is a sheath cover removably secured with a body of the sheath assembly.

In one aspect, the stem is rotated while being pushed towards the front end to allow outward expansion of the expandable member through the plurality of slots, to completely clean the inner surface of the catheter by removing any biofilm and killing harmful micro-organisms.

In one aspect, the front end is coated with an anticoagulant.

Another aspect of the present invention relates to a method for disinfecting inner surface of a catheter. The method includes inserting a device for disinfecting the catheter into the catheter, pushing the expandable member towards the front end by operating the stem to allow outward expansion of the expandable member through the plurality of slots when the expandable member reaches the front end, and pulling the device out of the catheter when the expandable member is present in the outwardly expanded form, thereby disinfecting the catheter.

In one aspect, the method includes rotating the device while pulling the device out of the catheter. Inserting the device into the catheter includes inserting the sheath assembly into any of an arterial line or a venous line of the catheter. The method also includes flushing the arterial line and the venous line of the catheter with a saline solution before inserting the device within, and removing residual saline solution from the arterial line and the venous line after pulling the device out of the catheter.

### BRIEF DESCRIPTION OF THE DRAWINGS

The accompanying drawings constitute a part of the description and are used to provide a further understanding of the present invention. The drawings illustrate exemplary embodiments of the present disclosure and, together with the description, serve to explain the principles of the present disclosure.

FIGS. 1A and 1B illustrate sectional side views of a device for disinfecting a catheter in inactivated and activated states respectively, in accordance with an embodiment of the present invention.

FIGS. 2A and 2B illustrate perspective side views of a device for disinfecting a catheter in inactivated and activated states respectively, in accordance with an embodiment of the present invention.

FIGS. 3A and 3B illustrate side views of a device for disinfecting a catheter being inserted into an arterial line and a venous line of the catheter, respectively, in accordance with an embodiment of the present invention.

FIG. 4 illustrates a perspective side view of a device for disinfecting a catheter fully inserted into an arterial line of the catheter, in accordance with an embodiment of the present invention.

FIG. 5 illustrates a device used for disinfecting a catheter disposed in a sterile packaging, in accordance with an embodiment of the present invention.

FIG. 6 illustrates a flowchart showing a method for disinfecting inner surface of a catheter, in accordance with an embodiment of the present invention.

### DETAILED DESCRIPTION OF THE INVENTION

The detailed description set forth below in connection with the appended drawings is intended as a description of various embodiments of the present invention and is not intended to represent the only embodiments in which the present invention may be practiced. Each embodiment described in this disclosure is provided merely as an example or illustration of the present invention, and should not necessarily be construed as preferred or advantageous over other embodiments. The detailed description includes specific details for the purpose of providing a thorough understanding of the present invention. However, it will be apparent to those skilled in the art that the present invention may be practiced without these specific details.

As used in the description herein, the meaning of “a,” “an,” and “the” includes plural reference unless the context clearly dictates otherwise. Also, as used in the description herein, the meaning of “in” includes “in” and “on” unless the context clearly dictates otherwise.

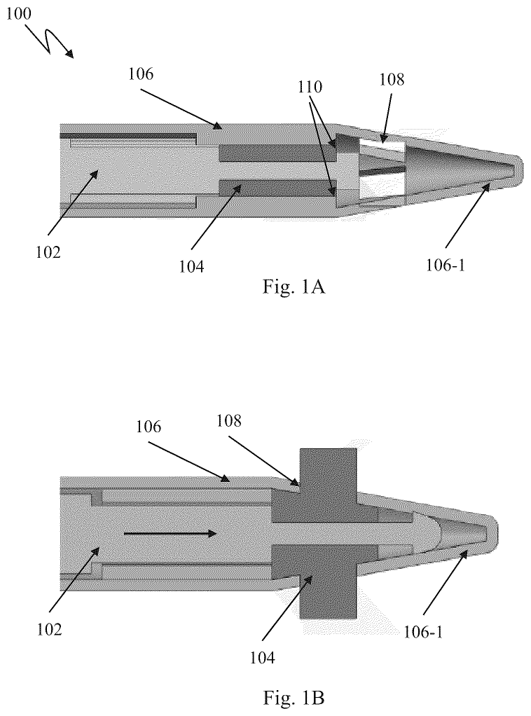

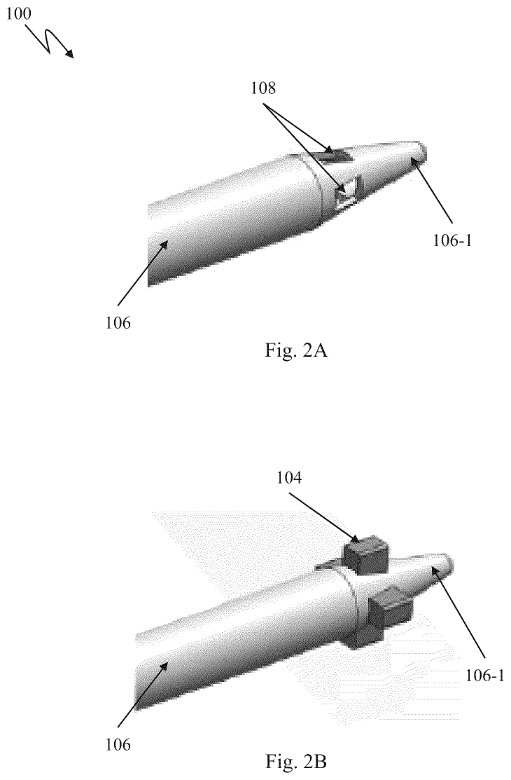

The present invention relates to a device for disinfecting and cleaning inner surface of a catheter to remove microbial contamination and prevent formation of fibrin sheath in the catheter, and a method thereof. FIGS. 1A and 1B illustrate sectional side views of a device 100 for disinfecting a catheter. FIGS. 2A and 2B illustrate perspective side views of the device 100 in inactivated and activated states respectively. The device 100 includes a stem 102 and an expandable member 104 fixed at a front end 102-1 of the stem 102. The expandable member 104 is wetted with a disinfectant, such as Povidone-iodine solution, saline solution, and citric acid. In certain implementations, citric acid of 0.5-1% concentration may be used as the disinfectant. The expandable member 104 may be selected as a polyurethane flexible foam having excellent absorption properties, when dipped with the disinfectant. Another criteria for selecting the expandable member 104 may be the flexibility/elasticity of the expandable member 104. The expandable member 104 is selected from a group consisting of a sponge, a foam, cotton, silicone rubber, polyurethane, or plastic mesh.

The device 100 also includes a sheath assembly 106 housing the stem 102 and the expandable member 104. The sheath assembly 106 includes a front end 106-1 that is closed, and a plurality of slots 108 present near the front end 106-1. The front end 106-1 is a sheath cover removably secured with a body of the sheath assembly 106. The front end 106-1 is coated with a suitable antithrombotic and antimicrobial coating to prevent thrombosis and catheter-related infection. The antithrombotic and antimicrobial coating may be prepared using Heparin, silver, chlorhexidine, rifampicine, or minocycline. In one implementation, the front end 106-1 may be coated with an anticoagulant, such as Heparin.

The plurality of slots 108 allow for outward expansion of the expandable member 104 when the expandable member 104 is pushed towards the front end 106-1 by operating the stem 102. Further, after a disinfection operation is completed, the expandable member 104 may be pulled away from the front end 106-1 and brought inward to come in a compressed state for safe disposal, by operating the stem 102.

In the inactivated state as shown in FIG. 2A, the expandable member 104 is present in a non-expanded/compressed position i.e. the expandable member 104 is not expanded out of the slots 108 of the sheath assembly 106. When the stem 102 is moved towards the front end 106-1 of the sheath assembly 106 (indicated by an arrow in FIG. 2B), the expandable member 104 wetted with the disinfectant expands by coming out of the slots 108 of the sheath assembly 106 and contacts the inner surface of the catheter, in the activated state. The device 100 may then be pulled away from the front end 106-1 of the sheath assembly 106 to enable disinfecting and cleaning of the inner surface of the catheter. Also, the expandable member 104 may be rotated, clockwise and/or counter clockwise, when the expandable member 104 is present in the activated state, for complete cleaning of the inner surface of the catheter, and remove any biofilm. Such manner of cleaning would prevent formation of fibrin sheath inside the catheter. The stem 102 may be removably provided such that the stem 102 is pushed into the sheath assembly 106 only during the procedure of disinfecting the catheter.

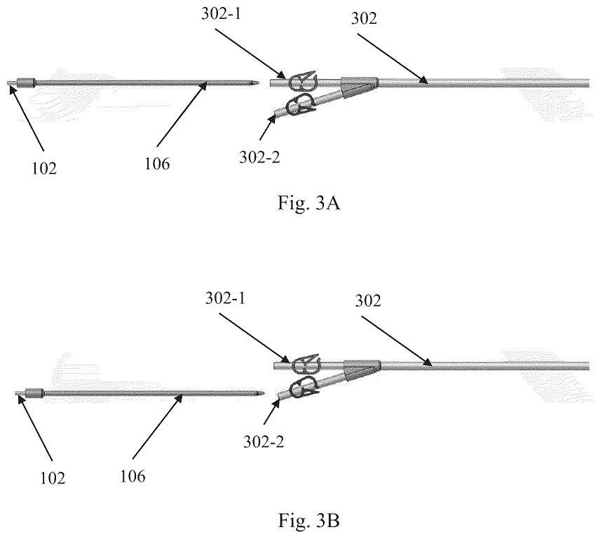

As shown in FIGS. 3A and 3B, the device 100 may be fully inserted into an arterial line 302-1 or a venous line 302-2 of a catheter 302, such as a high-flow dolphin catheter, to disinfect and clean the arterial line 302-1 or the venous line 302-2. The stem 102 may be pushed towards the front end 106-1 to enable outward expansion of the expandable member 104 wetted with the disinfectant, and contact the inner surface of the arterial line 302-1. The device 100 may then be pulled away from the front end 106-1 to enable disinfecting and cleaning of the inner surface of the catheter 302.

While the expandable member 104 is present in the activated state, the expandable member 104 may be rotated to completely clean and sanitize the inner surface of the arterial line 302-1 and prevent formation of fibrin sheath inside the arterial line 302-1. Further, as shown in FIG. 3B, the sheath assembly 106 may be inserted into the venous line 302-2 of the catheter 302, to disinfect and clean the venous line 302-2 in a similar manner. While pulling the device 100 away from the front end 106-1, the device 100 may be rotated to completely clean the inner surface of the catheter 302. This would remove any biofilm and prevent formation of fibrin sheath inside the catheter 302.

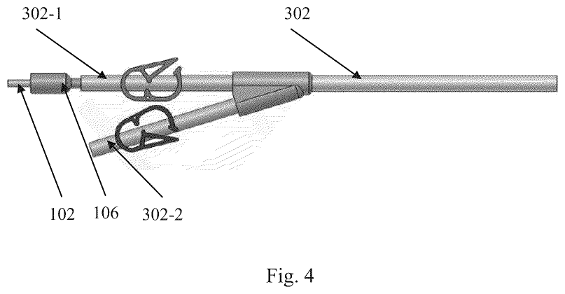

FIG. 4 illustrates a perspective view of the device 100 fully inserted into the arterial line 302-1 of the catheter 302. Length of the sheath assembly 106 of the device 100 may be larger than the catheter 302 in which the device 100 may need to be inserted. The sheath assembly 106 accommodating the stem 102 and the expandable member 104 may be inserted into any of the arterial line 302-1 or the venous line 302-2 of the catheter 302, such that the front end 106-1 of the sheath assembly 106 is in proximity of a tip of the catheter 302, to allow disinfection and cleaning of entire length of the catheter 302.

When the sheath assembly 106 accommodating the stem 102 and the expandable member 104 is inserted into the catheter 302, a rear portion of the stem 102 may extend beyond a rear portion of the sheath assembly 106, to allow a user to move the stem 102 towards the front end 106-1 of the sheath assembly 106 and gradually pulling the device 100 out of the catheter 302 for completing the disinfection procedure. It must be noted that the expandable member 104 cannot be pulled back into the sheath assembly 106 when the disinfection procedure is being carried out. Referring back to FIG. 1A, a front inner surface 110 (adjacent to the slots 108) of the sheath assembly 106 may act as a barrier for movement of the expandable member 104 within the sheath assembly 106, which may be caused due to accidental pulling back of the expandable member 104 during the disinfection procedure.

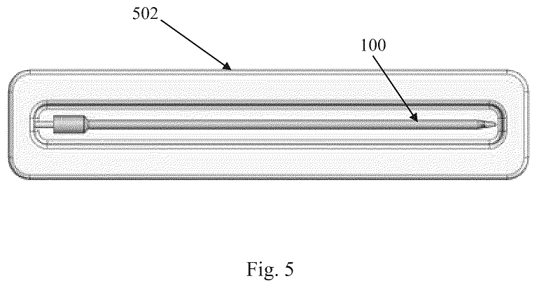

Once the disinfection procedure is completed, the expandable member 104 contaminated with biofilm or microbes may be pulled back with force into the sheath assembly 106, through the slots 108. Pulling of the expandable member 104 into the sheath assembly 106 would prevent the expandable member 104 from coming in contact with objects or surfaces outside the catheter 302, thereby preventing spreading of the contamination. Bringing the expandable member 104 back into the sheath assembly 106 after the disinfection procedure would help in safe disposal of the device 100, as the device 100 may be developed for single time use in certain implementations. Additionally, the device 100 may be enclosed in a sterile packaging 502 before disposal, as illustrated in FIG. 5. The sterile packaging 502 may be made of plastic, glass, or any other suitable material. Once closed, the sterile packaging 502 may become air locked and may not allow leakage of contaminants to the outside.

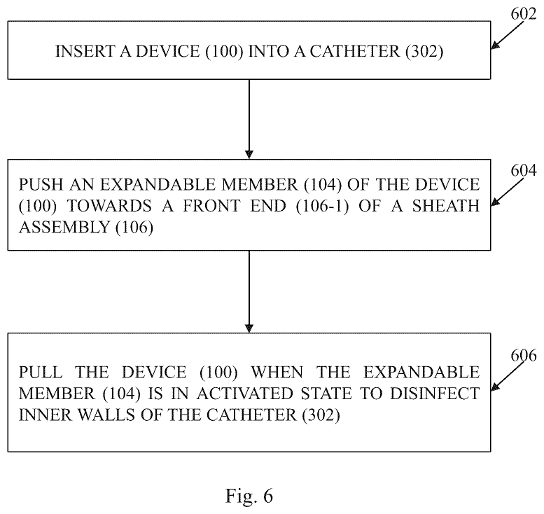

FIG. 6 illustrates a flowchart of a method for disinfecting the catheter, in accordance with an embodiment of the present invention. The method includes, at step 602, inserting a device 100 for disinfecting a catheter 302 into the catheter 302. The device includes a stem 102, an expandable member 104 fixed at a front end 102-1 of the stem 102, and a sheath assembly 106 housing the stem 102 and the expandable member 104. The expandable member 104 is wetted with a disinfectant. When the device 100 is inserted into the catheter 302, the sheath assembly 106 may be inserted into any of an arterial line 302-1 or a venous line 302-2 of the catheter 302 (such as a dolphin catheter).

As a precautionary measure, before inserting the sheath assembly 106 into the catheter 302, the catheter 302 may be flushed with a saline solution to prevent contact of residual blood which may be present in the catheter 302 with the device 100.

The method further includes, at step 604, pushing the expandable member 104 towards the front end 106-1 of the sheath assembly 106 by operating the stem 102, to allow outward expansion of the expandable member 104. A plurality of slots 108 are present near the front end 106-1 of the sheath assembly 106, which allow for outward expansion of the expandable member 104 through the slots 108 when the expandable member 104 is pushed towards the front end 106-1 by the stem 102 and reaches the front end 106-1.

The method includes, at step 606, pulling the device 100 out of the catheter 302 when the expandable member 104 is present in an activated state, i.e., in an outwardly expanded form. The device 100 is pulled away from the front end 106-1 of the sheath assembly 106 to enable disinfecting and cleaning of inner surface of the catheter 302, while the expandable member 104 is present in the activated state, so that the inner surface of the catheter 302 is completely disinfected and cleaned, and any residual biofilm present in the catheter 302 is removed. Also, while the device 100 is being pulled away from the front end 106-1, the device 100 may be rotated, either clockwise or counter clockwise, to complete clean the inner surface of the catheter 302 and prevent formation of fibrin sheath inside the catheter 302.

After the device 100 is pulled out of the catheter 302, any residual saline solution may be removed from the catheter 302 to remove traces of any residual biofilms. Thereafter, new injection line caps may be used to close inlet ports of the catheter 302.

Thus, the present invention provides a device for disinfecting and cleaning catheters, and a method thereof. The device is capable of capable of efficiently disinfecting inner surface of the catheters and removing microbial contamination of the catheters, to prevent bloodstream infections in patients. The device and the method described in present invention also prevent formation of fibrin sheath and traces of biofilm in the catheters. The device or one or more of its components may be replaced and/or disposed after cleaning and disinfecting a catheter, to ensure safety of the disinfection procedure. The device prevents usage of heparin lock solutions which typically imparts more weight on a catheter and increase pressure thereto. The device is easy to use and cost effective, while reducing operational time for disinfection of a catheter.

In the above detailed description, reference is made to the accompanying drawings that form a part thereof, and illustrate the best mode presently contemplated for carrying out the invention. However, such description should not be considered as any limitation of scope of the present unit. The structure thus conceived in the present description is susceptible of numerous modifications and variations, all the details may furthermore be replaced with elements having technical equivalence.

1. A device for disinfecting a catheter, comprising:
a stem;
an expandable member fixed at a front portion of the stem, wherein the expandable member is wetted with a disinfectant; and
a sheath assembly housing the stem and the expandable member wherein the sheath assembly includes:
a front end that is closed; and
a plurality of slots present near the front end wherein the plurality of slots allow for outward expansion of the expandable member when the expandable member is pushed towards the front end by the stem,
wherein when the sheath assembly is inserted within the catheter and the expandable member is brought outward by pushing through the stem, the expandable member wetted with the disinfectant comes in contact with an inner surface of the catheter thereby disinfecting the catheter.

2. The device as claimed in claim 1, wherein the expandable member is selected from a group consisting of sponge, foam, and cotton.

3. The device as claimed in claim 1, wherein the front end is a sheath cover removably secured with the sheath assembly.

4. The device as claimed in claim 1, wherein the front end is coated with an anticoagulant.

5. The device as claimed in claim 1, wherein the sheath assembly is inserted into any of an arterial line or a venous line of the catheter for disinfection.

6. A method for disinfecting a catheter the method comprising:
inserting a device for disinfecting the catheter into the catheter, the device comprising:
an expandable member fixed at a front portion of a stem and wetted with a disinfectant, and
a sheath assembly housing the stem and the expandable member,
wherein the sheath assembly includes a front end that is closed and a plurality of slots that are present near the front end;
pushing the expandable member towards the front end by operating the stem to allow outward expansion of the expandable member through the plurality of slots when the expandable member reaches the front end, wherein the expandable member wetted with the disinfectant and present in an outwardly expanded form makes contact with an inner surface of the catheter; and
pulling the device out of the catheter when the expandable member is present in the outwardly expanded form, thereby disinfecting the catheter.

7. The method as claimed in claim 6, comprising rotating the device while pulling the device out of the catheter.

8. The method as claimed in claim 6, wherein inserting the device into the catheter comprises inserting the sheath assembly into any of an arterial line or a venous line of the catheter.

9. The method as claimed in claim 8, comprising:
flushing the arterial line and the venous line of the catheter with a saline solution before inserting the device within; and
removing residual saline solution from the arterial line and the venous line after pulling the device out of the catheter.

10. The method as claimed in claim 6, wherein the expandable member is pulled back within the sheath assembly after completion of a disinfection procedure, for safe disposal of the device.
